(19) United States
(12) Patent Application Publication
(10) Pub. No.: US 2020/0038215 A1
(43) Pub. Date: Feb. 6, 2020

(54) TRANSIENT IMPLANTABLE DEVICE TO CONTROL ABSORPTION OF NUTRIENTS

(71) Applicant: Ethicon, Inc., Somerville, NJ (US)

(72) Inventor: An-Min Jason Sung, Warren, NJ (US)

(21) Appl. No.: 16/052,116

(22) Filed: Aug. 1, 2018

(51) Int. Cl. A61F 5/00

(52) U.S. Cl. CPC A61F 5/0076; A61F 5/0036

(57) ABSTRACT

A transient implantable device for placement within a gastrointestinal tract controls absorption of nutrients. The device includes a float that connects with a bypass tube. The device maintains its position within the gastrointestinal tract by floating on gastric fluid within the stomach. The device is further placed in a way where consumed food can pass around the device normally through the digestive tract, or food can pass through the device. The bypass tube includes a series of openings that are selectively obstructed by a film to control material entering and exiting from the bypass tube.

### BACKGROUND

In some instances, it may be desirable to deploy an endoluminal sleeve or other type of lining within a hollow body organ such as a stomach, intestine, etc. By way of example only, a sleeve may be positioned within a patient's duodenum to separate or bypass at least part of the food flow from the lined portions of the duodenum. In some patients, creating a physical barrier between ingested food and certain regions of the gastrointestinal wall by means of endoluminal sleeves may provide some degree of weight loss and/or treatment of type 2 diabetes. The presence of the barrier may influence or alter signaling (e.g., neural, endocrine, etc.) originating from the intestine and/or improve glycemic control. Contrary to traditional gastric bypass surgery, endoluminal sleeve surgery may be reversed and the sleeve may be removed after achievement of the desired clinical result.

An example of a duodenal sleeve is described in U.S. Pat. No. 7,267,694, entitled “Bariatric Sleeve,” issued Sep. 11, 2007, the disclosure of which is incorporated by reference herein. The proximal end of a flexible, floppy sleeve of impermeable material defining a sleeve lumen is endoscopically deployed and anchored with the help of a barbed stent in the pylorus or in the superior section of the duodenum. The stent is also intended to ensure that the proximal lumen opening of the sleeve remains open. Chyme from the stomach enters the proximal lumen opening of the sleeve and passes through the sleeve lumen to the distal lumen opening. Digestive enzymes secreted in the duodenum pass through the duodenum on the outside of the sleeve, with the sleeve isolating the chyme from the enzymes. The enzymes and the chyme do not mix until the chyme exits from the distal lumen opening of the liner sleeve. In such a way, the efficiency of the process of digestion of the chyme may be diminished, reducing the ability of the gastrointestinal tract to absorb calories from the food. The sudden exposure of chyme to the small intestine (e.g., duodenum, proximal jejunenum, etc.) at the distal end of the barrier may lead to altered signaling from the gastrointestinal system resulting in an improved metabolic response.

Additional examples of endoluminal sleeves, or endoluminal sleeve related devices, are disclosed in U.S. Pat. No. 7,121,283, entitled “Satiation Devices and Methods,” issued Oct. 17, 2006, the disclosure of which is incorporated by reference herein; U.S. Pat. No. 7,037,344, entitled “Apparatus and Methods for Treatment of Morbid Obesity,” issued May 2, 2006, the disclosure of which is incorporated by reference herein; U.S. Pub. No. 2008/0255678, entitled “Medical Apparatus and Method of Making the Same,” published Oct. 16, 2008, the disclosure of which is incorporated by reference herein; U.S. Pub. No. 2013/0030350, entitled “Devices and Methods for Anchoring an Endoluminal Sleeve in the GI Tract,” published Jan. 31, 2013, the disclosure of which is incorporated by reference herein; and U.S. Pub. No. 2017/0000638, entitled “Endoscopic Transoral Duodenal Sleeve Applier,” published Jan. 5, 2017, the disclosure of which is incorporated by reference herein. Yet another example of an endoluminal sleeve is the EndoBarrier® by G.I. Dynamics, Inc. of Watertown, Mass.

The above-noted examples of endoluminal sleeves include various structures and techniques to substantially maintain the position of the sleeve in the gastrointestinal tract. U.S. Pat. No. 7,931,693, entitled “Method and Apparatus for Reducing Obesity,” issued Apr. 26, 2011, the disclosure of which is incorporated by reference herein, discloses other structures and techniques that are used to substantially maintain the position of devices in the gastrointestinal tract. Similarly, U.S. Pat. No. 9,060,835, entitled “Conformationally-Stabilized Intraluminal Device for Medical Applications,” issued Jun. 23, 2015, the disclosure of which is incorporated by reference herein, and U.S. Pub. No. 2016/0135977, entitled “Conforming Anchor for Duodenal Barrier,” published May 19, 2016, the disclosure of which is incorporated by reference herein, also disclose structures and techniques that may be used to substantially maintain the position of devices in the gastrointestinal tract.

While a variety of metabolic related devices and have been made and used, it is believed that no one prior to the inventor(s) has made or used an invention as described in the appended claims.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements and in which:

FIG. 1 depicts a perspective view of an exemplary implantable device configured to control absorption of nutrients;

FIG. 2 depicts a cross-section view of the implantable device of FIG. 1, taken along line 2-2 of FIG. 1;

FIG. 3 depicts an enlarged view of the cross-section view of FIG. 2; and

FIG. 4 depicts a perspective view of the implantable device of FIG. 1, shown positioned within a stomach and a portion of the small intestine;

FIG. 5 depicts a partial perspective view of a tube usable with the implantable devices, showing opening with varying size along the tube;

FIG. 6 depicts a perspective view of another exemplary implantable device configured to control absorption of nutrients;

FIG. 7 depicts a cross-section view of the implantable device of FIG. 6, taken along line 7-7 of FIG. 6;

FIG. 8 depicts a partial perspective view of a proximal portion of the implantable device of FIG. 6, shown with the float members omitted to show connection members; and

FIG. 9 depicts a perspective view of another exemplary implantable device configured to control absorption of nutrients, showing fewer float members.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

### I. EXEMPLARY DEVICE WITH SINGLE SECTION FLOAT STRUCTURE

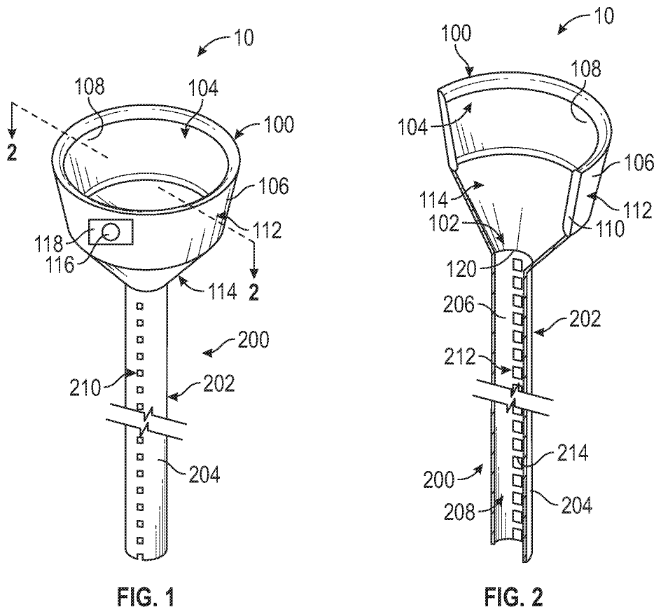

FIGS. 1 and 2 illustrate an exemplary implantable device (10), that is configured for use in a gastrointestinal tract. Device (10) can be implanted using an endoscopic procedure. Device (10) is further configured to mechanically impact the selection of the type of nutrients for absorption within the gastrointestinal tract, and further to reduce the overall intake of nutrients. Device (10) is further configured as a transient implantable device that can be self-expelled or removed endoscopically after some period of time.

Device (10) comprises a float (100) that is connectable with a bypass tube (200). In the illustrated version, float (100) comprises a funnel shape. In this manner, float (100) comprises a first opening (102) at a distal end of float (100), and a second opening (104) at a proximal end of float (100). In the present example where float (100) comprises a funnel shape, first opening (102) is smaller in diameter than second opening (104). In this configuration, second opening (104) is configured to receive food from a lower esophagus sphincter. First opening (102) is connectable with bypass tube (200) as described further below.

Float (100) comprises an outer sidewall (106) and an inner sidewall (108). Float (100) comprises a hollow interior (110) between outer sidewall (106) and inner sidewall (108), where hollow interior (110) defines a void volume that can be filled with a fluid. In the present version, hollow interior (110) is filled with air or another type of gas. With air-filled hollow interior (110), float (100) is operably configured to float on the gastric fluids within a stomach of the gastrointestinal tract. In view of the teaching herein, other suitable fluids for filling hollow interior (110) to achieve buoyancy within the stomach will be apparent to those of ordinary skill in the art.

In the example shown in FIGS. 1 and 2, float (100) comprises a single continuous section that extends in a circular manner. With this configuration, hollow interior (110) extends in a continuous fashion. Also in the illustrated version of FIGS. 1 and 2, float (100) comprises an upper region (112) and a lower region (114). As shown, hollow interior (110) is formed in upper region (112), and second opening (104) is defined by upper region (112). First opening (102) is defined by lower region (114). Moreover, as shown in the illustrated version of FIGS. 1 and 2, float (100) tapers more substantially in lower region (114) compared to upper region (112). Furthermore, the volume of float (100) can be lower in lower region (114) compared to upper regions (112) to maintain the funnel shape when device (10) is not under gastric pressure. In one version, this volume of float (100) in lower region (114) can be about 200 cubic centimeters, and even up to about 300 cubic centimeters in some versions. In other versions, float (100) can be configured without separate upper and lower regions (112, 114), or float (100) can be configured with a greater number of regions. Furthermore, the degree of tapering within the one or more regions used with float (100) can be different than that shown and described with respect to FIGS. 1 and 2, as will be apparent to those of ordinary skill in the art in view of the teachings herein.

Float (100) also comprises a port (116), which is positioned on a film (118). Port (116) is configured to permit air or another fluid to be added to or released from hollow interior (110) of float (100). In the present example, port (116) is self-sealing, e.g., a gel port, and further film (118) is an absorbable film. In this manner, float (100) is configured to deflate after a certain time once film (118) is absorbed sufficiently to yield a loss in film's (118) integrity such that the seal film (118) makes with hollow interior (110) of float (100) is broken. When this occurs, float (100) deflates and loses its buoyancy, such that it may now be self-expelled from the gastrointestinal tract or removed endoscopically more easily. Additionally, when positioning or placing device (10) within the stomach of a patient, port (116) allows device (10) to be positioned in an uninflated state and then once within the stomach, air or another fluid can be added via port (116) such that float (100) is changed to an inflated state.

As mentioned above, first opening (102) of float (100) is connectable with bypass tube (200). In the present example, float (100) and bypass tube (200) are connectable using absorbable suture (120). In this manner, the material for suture (120) can be designed and selected with a desired time for resorption such that after the desired time has passed and suture has resorbed, bypass tube (200) can separate from float (100) to aid in removal of device (10) by self-expulsion or endoscopically. Still in other versions, suture (120) can be configured to make a permanent connection between bypass tube (200) and float (100).

Bypass tube (200) comprises an elongated body (202) having an outer sidewall (204), an inner sidewall (206), and a hollow interior (208) defined by inner sidewall (206). In the present example, bypass tube (200) further comprises a plurality of openings (210) that extend between and through outer and inner sidewalls (204, 206) creating a passage, channel, or access from outside bypass tube (200) to hollow interior (208) of bypass tube (200).

Bypass tube (200) has a proximal end that connects with float (100) as discussed above, and a distal end of bypass tube (200) extends away from float (100). In the illustrated examples of FIGS. 1 and 2, openings (210) extend along a length of bypass tube (200) from its proximal end to its distal end. As also shown in the illustrated examples of FIGS. 1 and 2, openings (210) are arranged on two opposing sides of bypass tube (200). In this manner, openings (210) comprise a first longitudinally extending row on a first side of bypass tube (200), and a second longitudinally extending row on an opposite second side of bypass tube (200). With bypass tube's (200) cylindrical and tubular shape, these two rows of openings are spaced radially about 180 degrees apart around a circumference of bypass tube (200). In view of the teachings herein, other arrangements for plurality of openings (210) that may be used with bypass tube (200) will be apparent to those of ordinary skill in the art. For instance, the spacing between openings (210) can be altered longitudinally and radially as desired.

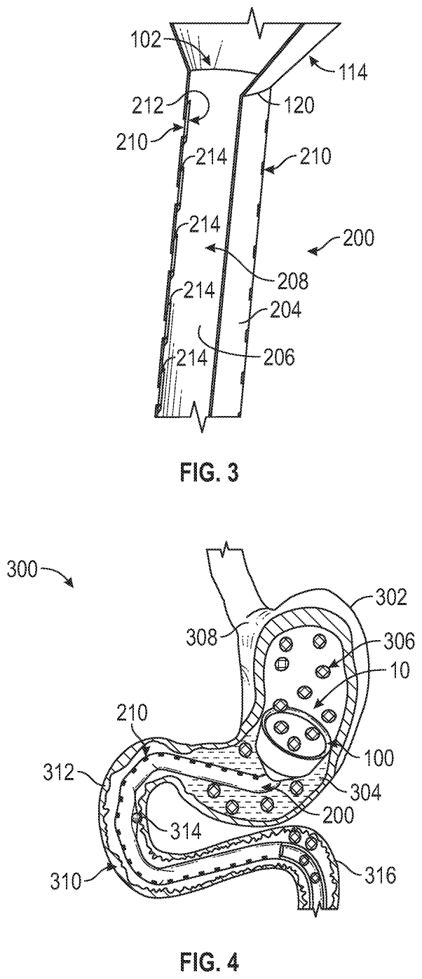

Referring now to FIGS. 2 and 3, Bypass tube (200) further comprises a plurality of curtains (212) that are each configured to attach with a part of inner sidewall (206) of bypass tube (200). Each curtain (212) is further configured to cover or extend over one of plurality of openings (210). In this manner, each curtain (212) selectively covers, closes, or blocks its associated opening (210). Moreover, curtains (212) are connected with inner sidewall (206) along a proximal end (214) of curtain (212), with the remaining portion of curtain (212) draping or extending distally to cover its associated opening (210). Furthermore, in the present example, curtains (212) are sized larger than openings (210) to ensure full coverage over openings (210). In the present example, curtains (212) are comprised of a thin film material that may be bonded to inner sidewall (206) of bypass tube (200) along proximal end (214) as described above.

In this arrangement for openings (210) and curtains (212), when food travels through bypass tube (200) from the proximal end to the distal end, curtains (212) cover openings (210) such that food cannot escape from bypass tube (200) out openings (210). In this way, the combination of openings (210) with curtains (212) define one-way openings or passages that keep food within bypass tube (200) when food travels along the digestive pathway from top down. However, when food travels in the opposite direction—from the distal end to the proximal end of bypass tube (200), which generally coincides with a bottom up direction along the digestive pathway—food is able to exit the bypass tube (200) through openings (210). This may occur, e.g., under repulsive forces from a pyloric sphincter. When this occurs, upward forces acting within bypass tube (200) push or lift curtains (212) to thereby provide access to openings (210) such that food and other digestive material can pass from within bypass tube (200) through openings (210) to outside of bypass tube (200).

Additionally, with the above-described configuration where curtains (212) are positioned along inner sidewall (206) along the inside of bypass tube (200), material outside of bypass tube (200) can flow into bypass tube (200) through openings (210) based on the one-way openings or passages defined by the combined openings (210) and curtains (212). This is the case whether the material outside bypass tube (200) is moving from top down or from bottom up within the digestive pathway or gastrointestinal tract. When this occurs, the material initially outside bypass tube (200), can push on curtains (212) to gain access to inside bypass tube (200) since curtains (212) are connected with inner sidewall (206) of bypass tube (200) only along one side. And with curtains (212) bonded to bypass tube (200) along their proximal end (214), curtains (212) can generally guide or direct the outside entering material in a distal direction within bypass tube (200). It will be apparent to those of ordinary skill in the art in view of the teachings herein that the material within the gastrointestinal tract that is initially outside bypass tube (200) can include, but is not limited to, food, gastric fluid, and digestive enzymes. It will also be apparent to those of ordinary skill in the art in view of the teachings herein that the terms gastrointestinal tract and digestive tract and digestive pathway may be used interchangeably.

By way of example and not limitation, for portions of bypass tube (200) residing in the intestine, liquid digestive material, including but not limited to pancreatic juice and other hormones, can flow from outside bypass tube (200) to within bypass tube (200) through openings (210) by pushing through curtains (212) under peristalsis motion. In this manner, device (10) is configured to allow pre-mixing of digestive fluids, including but not limited to intrinsic factor (IF), with the lipids, B12, and other oil soluble vitamins for normal absorption in the ileum.

Referring to FIG. 4, device (10) is shown positioned within a portion of a gastrointestinal tract (300). As shown, float (100) is located within a stomach (302) and floats upon gastric fluid (304) within stomach (302). In this manner, float (100) will move up and down depending on the level of gastric fluid (304) within stomach (302). Float (100) is configured such that it is not a tight fit within stomach (302). Therefore, food (306) that enters from lower esophagus sphincter (308) will not all fall into or be caught or captured by funnel-shaped float (100), particularly with the gastric motility. Thus, some food (306) will follow the normal digestive pathway and bypass device (10) entirely. The portion of food (306) that enters the funnel-shaped float (100) and travels through device (10) will be digested slower since the amount of acid will be lower. This prolongs the satiety feeling in addition to the volume of float (100) itself contributing to the satiety feeling. The volume of float (100) itself contributing to the satiety feeling mimics the effect of other restrictive procedures. The portion of food (306) that passes normally through the digestive tract, and not through device (10), can pass normally by the duodenal papilla to signal secretion of cholecystokinin, secretin, and gastrin as in the normal digestive process. In addition, the partial passage of food normally through the duodenum section minimizes the malabsorption of water soluble vitamins and minerals. Thus device (10) is configured to not entirely block absorption at the duodenum section, sometimes herein referred to as the front gut, but instead to partially block absorption at the duodenum section.

Bypass tube (200), as shown in FIG. 4, extends from a lower portion of stomach (302) and into a portion of small intestine (310). Bypass tube (200) extends such that it covers or extends past the duodenum (312) and past the duodenal papilla (314). In some versions, bypass tube (200) can extend even further along gastrointestinal tract (300). For instance, as shown in FIG. 4 but not required in all versions, bypass tube (200) extends to part of a jejunum (316).

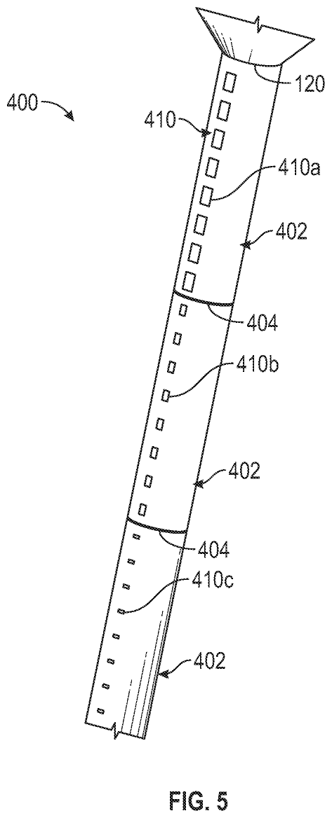

In configuring device (10) for a desired application, and in particular the length of bypass tube (200) desired, in some versions bypass tube (200) is comprised of multiple sections that are joined or joinable together. Referring to FIG. 5, in one version of a bypass tube (400) usable with float (100) of device (10), multiple sections (402) of bypass tube (400) are joined together using absorbable suture (404). Suture (404) is configurable the same or similar to suture (120) described above, and like suture (120), suture (404) can be resorbed over time such that sections (402) may separate or slough off to aid in removal or self-expulsion of all or part of device (10). In some instances, suture (404) can have varying breakdown or resorption time depending on the location of sections (402) being joined. For instance, suture (404) that attaches the distal most section (402) with its adjacent section (402) may be configured to breakdown and resorb quickest such that when it is time for device (10) to be removed or expelled, small portions separate or slough off rather than device (10) breaking into large parts that may be more difficult to remove or self-expel. Thus, in this manner, suture (404) can be configured to minimize or aid in minimizing the removal complexity of device (10). Of course, in other versions, suture (404) may be permanent material that is not configured to be resorbed over time. In view of the teachings herein, various ways to configure device (10) to provide the desired bypass tube length will be apparent to those of ordinary skill in the art.

The size of openings (210) of bypass tube (200) can be configured to control or influence digestion and absorption. For example, in one version, openings (210) of bypass tube (200) can have a size larger than about 3 millimeters up to about 10 millimeters. In some versions, the size of openings (210) will not exceed 10 millimeters. In some versions, the size of openings (210) can be larger than about 3 millimeters up to about 10 millimeters for the portion of bypass tube (200) residing or positioned in the stomach, and smaller than about 3 millimeters for the portion of bypass tube (200) residing or positioned in the intestine. In some instances, the size of openings (210) can vary. In such version with varying sized openings (210), for the portion of bypass tube (200) residing or positioned in the intestine, openings (210) can be smaller than about 3 millimeters, even down to a slit. Referring again to FIG. 5, bypass tube (400) shows one version of a bypass tube that has varying sized openings (410). More specifically, bypass tube (400) has larger sized openings (410a) near its proximal end, slots or slot-sized openings (410c) near its distal end, and intermediate sized openings (410b) between larger openings (410a) and slots (410c). In view of the teachings herein, other configurations for the size of openings (210, 410) will be apparent to those of ordinary skill in the art.

When considering an exemplary use of device (10) after device (10) has been placed within a patient, food like carbohydrates that are easily digested will be liquefied at an early stage between a mouth and the stomach. A portion of this will flow directly through the funnel-shaped float (100) to bypass tube (200), and not be absorbed at the duodenum. However, another portion of this will not enter device (10) and instead travel the normal digestive pathway or tract. With some of these easily digested carbohydrates not being absorbed, this will lower the glycemic index. In this manner, the portion of bypass tube (200) in the intestine is configured to prevent food within device (10) from being absorbed by the intestine, thus device (10) mimics the effect of malabsorption procedures.

Foods like proteins and lipids particulates that are larger than about 3 millimeters, will remain in the stomach, either inside the funnel-shaped float (100) or outside float (100) due to the filtering function of the pyloric sphincter until they are digested. Those protein and lipid particulates that are smaller than about 3 millimeters, and that enter the funnel-shaped float (100), will travel down bypass tube (200) and arrive to the distal gut early to trigger the secretion of glucagon-like peptide-1 (GLP1) to adjust the insulin level. This can contribute to the relief of type 2 diabetes (T2D) symptoms soon after using device (10), similar to the Roux-en-Y gastric bypass procedure (RXY procedure).

Thus in at least some ways, device (10) is configured to mimic the effect achieved by the RXY procedure without removal or rearranging anatomical structures like in the RXY procedure. Additionally, because device (10) is configured as a transient solution, where device (10) can be either self-expelled or removed in an endoscopic procedure, device (10) is configured to avoid complications that can be associated with more invasive surgeries and/or implantable devices. For instance, device (10) lacks any mechanical anchor to tissues in either the stomach or intestine, which can cause bleeding or discomfort.

### II. EXEMPLARY DEVICE WITH MULTI-SECTION FLOAT STRUCTURE

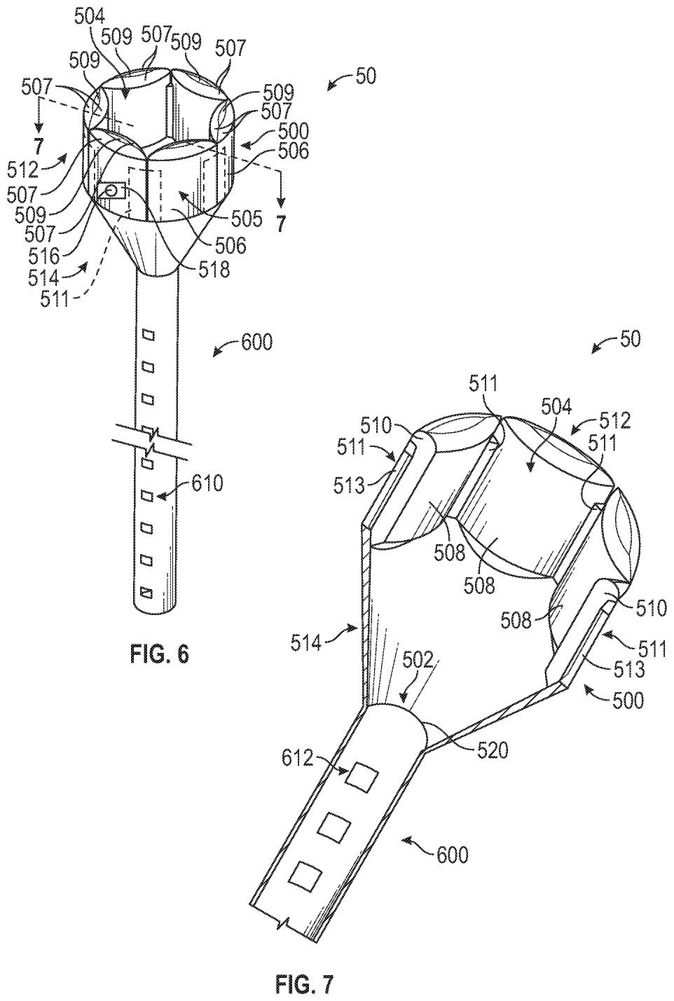

Referring to FIGS. 6-8, another exemplary implantable device (50) is shown. Device (50) comprises float (500) and bypass tube (600). Bypass tube (600) comprises a plurality of openings (610) and also a plurality of curtains (612) the same as openings (210) and curtains (212) described above. In one version of device (50), bypass tube (600) is the same as bypass tube (200) described above, including all shown and described features of bypass tube (200). In another version of device (50), bypass tube (600) is the same as bypass tube (400) described above, including all shown and described features of bypass tube (400). Having previously described bypass tubes (200, 400) in detail, it will be understood that all such details apply equally to bypass tube (600) and that no further duplicative description is necessary to fully describe bypass tube (600). Furthermore, in view of the teachings herein, various modifications to bypass tube (600) are possible and such modifications will be apparent to those of ordinary skill in the art in view of the teachings herein.

Float (500) of device (50) is similar to float (100) of device (10) in certain respects. For example, float (500) is connectable with a bypass tube (600) in the same manner that float (100) is connectable with bypass tube (200). In the illustrated version, float (500) comprises a funnel shape. In this manner, float (500) comprises a first opening (502) at a distal end of float (500), and a second opening (504) at a proximal end of float (500). In the present example where float (500) comprises a funnel shape, first opening (502) is smaller in diameter than second opening (504). In this configuration, second opening (504) is configured to receive food from a lower esophagus sphincter. First opening (502) is connectable with bypass tube (600).

Float (500) differs from the illustrated version of float (100) in at least that float (500) comprises multiple sections (505) that are positioned in a circular arrangement defining a circumference of float (500). Each section (505) comprises an outer sidewall (506) and an inner sidewall (508). In their circular arrangement, first opening (502) is defined by sections (505). Each section (505) further comprises a pair of shoulders (507) that extend upward and angularly toward each other before meeting a top cap portion (509). Each section (505) of float (500) comprises a hollow interior (510) between outer sidewall (506) and inner sidewall (508), where hollow interior (510) defines a void volume that can be filled with a fluid. In the present version, hollow interior (510) is filled with air or another type of gas. With air-filled hollow interior (510), float (500) is operably configured to float on the gastric fluids within a stomach of the gastrointestinal tract. In view of the teaching herein, other suitable fluids for filling hollow interior (510) to achieve buoyancy within the stomach will be apparent to those of ordinary skill in the art.

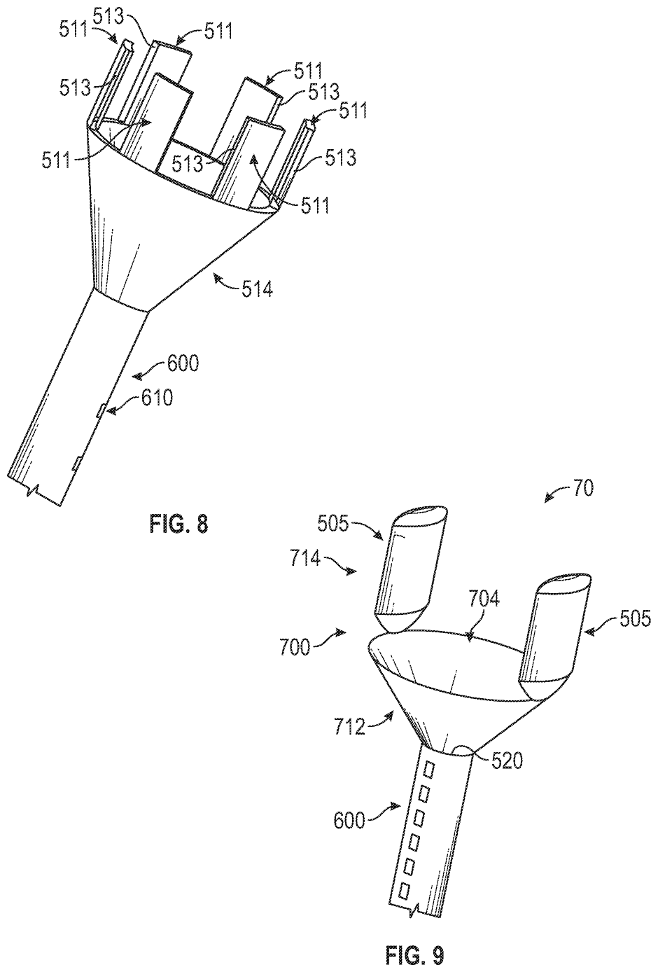

In the example shown in FIGS. 6-8 with multiple sections (505) arranged in a circular manner, hollow interiors (510) extend in a circular fashion. Furthermore, connecting members (511) with hollow interiors (513) are located between each section (505) and extend within each section (505). In this manner, a continuous hollow channel or chamber is defined by sections (505) and connecting members (511) that extends around a perimeter of float (500). FIG. 8 shows a partial view of float (500) without sections (505) to reveal more clearly connecting members (511) and their hollow interiors (513). As shown in the present example of FIG. 8, connecting members (511) connect with lower region (514) of float (500) and extend upward or proximally. FIG. 6 further shows some of connecting members (511) located within sections (505) in phantom, however a portion of some connecting members (511) is further seen from the interior of float (500) as FIG. 7 more clearly shows.

Also in the illustrated version of FIGS. 6 and 7, float (500) comprises an upper region (512) and a lower region (514). As shown, hollow interior (510) is formed in upper region (512), and second opening (504) is defined by upper region (512). First opening (502) is defined by lower region (514). Moreover, as shown in the illustrated version of FIGS. 6 and 7, float (500) tapers more substantially in lower region (514) compared to upper region (512). Furthermore, the volume of float (500) can be lower in lower region (514) compared to upper regions (512) to maintain the funnel shape when device (50) is not under gastric pressure. In one version, this volume of float (500) in lower region (514) can be about 200 cubic centimeters, and even up to about 300 cubic centimeters in some versions. In other versions, float (500) can be configured without separate upper and lower regions (512, 514), or float (500) can be configured with a greater number of regions. Furthermore, the degree of tapering within the one or more regions used with float (500) can be different than that shown and described with respect to FIGS. 6 and 6, as will be apparent to those of ordinary skill in the art in view of the teachings herein.

Float (500) also comprises a port (516), which is positioned on a film (518). Port (516) is configured to permit air or another fluid to be added to or released from the continuous hollow channel formed by hollow interiors (510) of sections (505) and hollow interiors (513) of connecting members (511). In the present example, port (516) is self-sealing, e.g., a gel port, and further film (518) is an absorbable film. In this manner, float (500) is configured to deflate after a certain time once film (518) is absorbed sufficiently to yield a loss in film's (518) integrity such that the seal film (518) makes with the continuous hollow channel is broken. When this occurs, float (500) deflates and loses its buoyancy, such that it may now be self-expelled from the gastrointestinal tract, or more easily be removed endoscopically. Additionally, when positioning or placing device (50) within the stomach of a patient, port (516) allows device (50) to be positioned in an uninflated state and then once within the stomach, air or another fluid can be added via port (516) such that float (500) is changed to an inflated state.

As mentioned above, float (500) is configured with multiple sections (505) and having a connected air channel created by the continuous hollow channel, which is defined by hollow interiors (510) of sections (505) and hollow interiors (513) of connecting members (511). With this configuration, float (500) is configured to be collapsible. For instance, in the illustrated version of FIGS. 6 and 7, in some cases shoulders (507) and top cap portions (509) are configured to collapse to comply with the pressure from gastric motility, yet maintain the funnel shape of float (500) to catch foods. In this fashion, shoulders (507) and top cap portions (509) are considered the collapsible point of float (500). In other versions, other portions of float (500) may instead or in addition be consider collapsible points of float (500). For example, each individual section (505) itself can be at least somewhat collapsible in some versions.

When portions of float (500) collapse, this collapsing action allows a redistribution of the air or fluid within the continuous hollow channel such that the collapsing portion of float (500) can be responsive to the pressure from gastric motility. In this manner, collapsible does not require a loss of air or fluid from the continuous hollow channel. Furthermore, when pressures from gastric motility reduce, the collapsible portions of float (500) return to their initial positions based on a uniform distribution of air or fluid within the connected air channel defined by the continuous hollow channel as described above. Still, in some versions, port (516) may be configured to allow some release of air or fluid under certain circumstances, or when certain pressures are reached within the stomach.

As mentioned above, first opening (502) of float (500) is connectable with bypass tube (600). In the present example, float (500) and bypass tube (600) are connectable using absorbable suture (520). In this manner, the material for suture (520) can be designed and selected with a desired time for resorption such that after the desired time has passed and suture has resorbed, bypass tube (600) can separate from float (500) to aid in removal of device (50) by self-expulsion or endoscopically. Still in other versions, suture (520) can be configured to make a permanent connection between bypass tube (600) and float (500).

Another feature of floats (100, 500) is that the funnel shape spreads the occupying volume of floats (100, 500) longitudinally around the circumference of the stomach. This configuration can help minimize the intolerance patients have to the implanted device (10, 50) compared to other types of gastric implants, such as balloons, etc. This intolerance can be even further minimized where the float is collapsible or has collapsible portions, such as with float (500) described above so that device (50) more easily and naturally responds to pressure from gastric motility.

### III. EXEMPLARY DEVICE WITH REDUCED MATERIAL USAGE

Referring to FIG. 9, another exemplary device (70) is shown that reduces the amount of material usage for the implant by reducing the size of the float components. More specifically, device (70) comprises float (700) and bypass tube (600). Float (700) is connectable with bypass tube (600) as described above and in the same manner that float (500) is connectable with bypass tube (600). In the illustrated version, float (700) comprises a funnel shape as described above with respect to floats (100, 500); however, the majority of the funnel shape with float (700) is provided in a lower region (712). In this manner, float (700) comprises a first opening at a distal end of float (700), and a second opening (704) at a proximal end of float (700). In the present example where float (700) comprises a funnel shape, the first opening is smaller in diameter than second opening (704). In this configuration, second opening (704) is configured to receive food from a lower esophagus sphincter, while the first opening is connectable with bypass tube (600).

Float (700) comprises multiple sections (505), however, float (700) differs from the illustrated version of float (500) in at least that float (700) comprises fewer sections (505) compared with float (500). For instance, the illustrated version depicts only two sections (505); however, other arrangements using more or fewer sections (505) is possible and will be apparent to those of ordinary skill in the art in view of the teachings herein. Also, with device (70) and float (700), because of the fewer sections (505), connecting members (511) are omitted in the illustrated version of FIG. 9. Thus, with the illustrated version of FIG. 9, each section (505) includes hollow interior (510) as described above; however, hollow interiors (510) are not interconnected directly or indirectly by connecting members (511) like with float (500).

In the example shown in FIG. 9, float (700) comprises an upper region (712) and a lower region (714). Upper region (712) comprises a pair of sections (505) that connect with lower region (712) and are generally positioned opposite each other. In the present example, sections (505) connect with lower region (712) of float (700) and are not impeded by any other sections adjacent to sections (505). Thus, sections (505) are responsive to pressure from gastric motility, while also being configured to provide buoyancy such that device (70) floats on gastric fluid within the stomach. Therefore, in this manner float (700) is configured to be collapsible based on the ability for sections (505) to move or shift in response to pressure from gastric motility.

Either or both sections (505) of float (700) can comprises a port (516), which is positioned on a film (518) as described above with respect to sections (505) of device (50). Port (516) is configured to permit air or another fluid to be added to or released from the hollow interiors (510) of sections (505). In the present example, port (516) is self-sealing, e.g., a gel port, and further film (518) is an absorbable film. In this manner, where float (700) includes port (516) and film (518), float (700) is configured to deflate after a certain time once film (518) is absorbed sufficiently to yield a loss in film's (518) integrity such that the seal film (518) makes with sections (505) is broken. When this occurs, float (700) deflates and loses its buoyancy such that it may now be self-expelled from the gastrointestinal tract or removed endoscopically more easily. Additionally, when positioning or placing device (70) within the stomach of a patient, port (516) allows device (70) to be positioned in an uninflated state and then once within the stomach, air or another fluid can be added via port (516) such that float (700) is changed to an inflated state.

As mentioned above, the first opening of float (700) is connectable with bypass tube (600). In the present example, float (700) and bypass tube (600) are connectable using absorbable suture (520). In this manner, the material for suture (520) can be designed and selected with a desired time for resorption such that after the desired time has passed and suture has resorbed, bypass tube (600) can separate from float (700) to aid in removal of device (70) by self-expulsion or endoscopically. Still in other versions, suture (520) can be configured to make a permanent connection between bypass tube (600) and float (700).

### IV. EXEMPLARY COMBINATIONS

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

A gastrointestinal implant device configured for controlling absorption of nutrients, comprises: (a) a float configured to be positioned within a stomach and selectively remain within the stomach by buoyant forces acting on the float, wherein the float comprises: (i) an outer sidewall, (ii) an inner sidewall, (iii) a hollow interior defined between the outer sidewall and the inner sidewall, wherein the hollow interior is configured to hold a fluid, (iv) a first opening at a distal end of the float, and (v) a second opening at a proximal end of the float, wherein the second opening is configured to receive consumed food from a lower esophagus sphincter; and (b) a bypass tube configured to connect with the float at the distal end of the float, wherein the bypass tube comprises a plurality of openings.

### Example 2

The device of Example 1, wherein the float further comprises a self-sealable port in fluid communication with the hollow interior, wherein the port is configured to allow fluid to be added or removed from the float.

### Example 3

The device of Example 2, wherein the port is formed on an absorbable film.

### Example 4

The device of any one or more of Examples 1 through 3, wherein the float comprises a funnel shape.

### Example 5

The device of any one or more of Examples 1 through 4, wherein the float further comprises two or more sections.

### Example 6

The device of any one or more of Examples 1 through 5, wherein a portion of the hollow interior of the float is defined by two or more sections.

### Example 7

The device of any one or more of Examples 1 through 6, wherein the float further comprises two or more sections, wherein the sections are each collapsible.

### Example 8

The device of any one or more of Examples 1 through 7, wherein the size of the plurality of openings of the bypass tube varies.

### Example 9

The device of any one or more of Examples 1 through 8, wherein the plurality of openings of the bypass tube extend in a manner selected from longitudinally along a length of the bypass tube, radially around a circumference of the bypass tube, or a combination thereof.

### Example 10

The device of any one or more of Examples 1 through 9, wherein the device is configured to fit with the stomach such that the second opening of the float is configured to receive a first portion of the consumed food, while the device is configured to permit a second portion of the consumed food to bypass the second opening of the float to pass through the stomach normally.

### Example 11

The device of any one or more of Examples 1 through 10, wherein the bypass tube is selectively mechanically fastened to the float with absorbable material.

### Example 12

The device of any one or more of Examples 1 through 11, wherein the bypass tube comprises multiple sections that are selectively joinable by mechanical fastening with absorbable material.

### Example 13

The device of any one or more of Examples 1 through 12, wherein the bypass tube is configured to extend within a gastrointestinal tract such that the duodenum region past the duodenal papilla is covered by the bypass tube.

### Example 14

The device of any one or more of Examples 1 through 13, wherein the bypass tube is configured to extend within a gastrointestinal tract to part of a jejunum of the gastrointestinal tract.

### Example 15

The device of any one or more of Examples 1 through 14, further comprising a plurality of curtains that are connectable with an inner sidewall of the bypass tube, wherein each of the plurality of curtains are configured to selectively cover one of the plurality of openings.

### Example 16

The device of any one or more of Examples 1 through 15, wherein the bypass tube is configured to permit digestive fluid to flow into the bypass tube through one or more of the plurality of openings under peristalsis motion.

### Example 17

A gastrointestinal implant device is configured for controlling absorption of nutrients. The device comprises (a) a float configured to be positioned within a stomach and selectively remain within the stomach by buoyant forces acting on the float, wherein the float comprises: (i) two or more sections, wherein each of the sections comprise a first hollow interior configured to hold a fluid, (ii) one or more connecting members, wherein each of the connecting members comprise a second hollow interior, wherein the one or more connecting members are configured to establish fluid communication between the two or more sections, such that the first hollow interior of each section in combination with the second hollow interior of each connecting member defines a continuous fluid channel, (iii) a first opening at a distal end of the float, and (iv) a second opening at a proximal end of the float, wherein the second opening is configured to receive consumed food from a lower esophagus sphincter; and (b) a bypass tube configured to connect with the float at the distal end of the float, wherein the bypass tube comprises a plurality of openings and a plurality of curtains, wherein each of the curtains is operably configured to selectively cover one of the plurality of openings.

### Example 18

The device of Example 17, wherein the sections and connecting members are located adjacent one another in an alternating fashion thereby defining a continuous perimeter of the float.

### Example 19

The device of any one or more of Examples 17 through 18, wherein the continuous fluid channel is filled with air.

### Example 20

A method for assembling a device to control absorption of nutrients within a gastrointestinal tract using a transient implantable device, the method comprising: (a) connecting a float of the device having a funnel shape with a bypass tube, wherein the float is configured to float upon gastric fluid within a stomach of the gastrointestinal tract; (b) creating a plurality of openings within the bypass tube, such that the plurality of openings extend though an outer sidewall and an inner sidewall of the bypass tube to a hollow interior of the bypass tube, wherein the plurality of openings are spaced longitudinally along the bypass tube; and (c) connecting a plurality of curtains to the bypass tube, wherein each of the plurality of curtains is configured to cover one of the plurality of openings, wherein the combination of the plurality of openings and the plurality of curtains together define a plurality of one-way openings along the bypass tube, wherein the plurality of one-way openings are configured to restrict food passing through the bypass tube from a proximal end to a distal end from exiting through any one of the plurality of one-way openings, wherein the plurality of one-way openings are further configured to permit digestive fluid within an intestine of the gastrointestinal tract to enter into the hollow interior of the bypass tube from outside of the bypass tube under peristalsis motion.

### V. MISCELLANEOUS

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions of the devices described above may have application in conventional medical treatments and procedures conducted by a medical professional, as well as application in robotic-assisted medical treatments and procedures. By way of example only, various teachings herein may be readily incorporated into a robotic surgical system such as the DAVINCI™ system by Intuitive Surgical, Inc., of Sunnyvale, Calif.

Versions of the devices described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility, or by a user immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. A gastrointestinal implant device configured for controlling absorption of nutrients, the device comprising:
(a) a float configured to be positioned within a stomach and selectively remain within the stomach by buoyant forces acting on the float, wherein the float comprises:
(i) an outer sidewall,
(ii) an inner sidewall,
(iii) a hollow interior defined between the outer sidewall and the inner sidewall, wherein the hollow interior is configured to hold a fluid,
(iv) a first opening at a distal end of the float, and
(v) a second opening at a proximal end of the float, wherein the second opening is configured to receive consumed food from a lower esophagus sphincter; and
(b) a bypass tube configured to connect with the float at the distal end of the float, wherein the bypass tube comprises a plurality of openings.

2. The device of claim 1, wherein the float further comprises a self-sealable port in fluid communication with the hollow interior, wherein the port is configured to allow fluid to be added or removed from the float.

3. The device of claim 2, wherein the port is formed on an absorbable film.

4. The device of claim 1, wherein the float comprises a funnel shape.

5. The device of claim 1, wherein the float further comprises two or more sections.

6. The device of claim 5, wherein a portion of the hollow interior of the float is defined by each of the sections.

7. The device of claim 5, wherein the sections are each collapsible.

8. The device of claim 1, wherein the size of the plurality of openings of the bypass tube varies.

9. The device of claim 1, wherein the plurality of openings of the bypass tube extend in a manner selected from longitudinally along a length of the bypass tube, radially around a circumference of the bypass tube, or a combination thereof.

10. The device of claim 1, wherein the device is configured to fit with the stomach such that the second opening of the float is configured to receive a first portion of the consumed food, while the device is configured to permit a second portion of the consumed food to bypass the second opening of the float to pass through the stomach normally.

11. The device of claim 1, wherein the bypass tube is selectively mechanically fastened to the float with absorbable material.

12. The device of claim 1, wherein the bypass tube comprises multiple sections that are selectively joinable by mechanical fastening with absorbable material.

13. The device of claim 1, wherein the bypass tube extends within a gastrointestinal tract such that the duodenum region past the duodenal papilla is covered by the bypass tube.

14. The device of claim 1, wherein the bypass tube is configured to extend within a gastrointestinal tract to part of a jejunum of the gastrointestinal tract.

15. The device of claim 1, further comprising a plurality of curtains that are connectable with an inner sidewall of the bypass tube, wherein each of the plurality of curtains are configured to selectively cover one of the plurality of openings.

16. The device of claim 1, wherein the bypass tube is configured to permit digestive fluid to flow into the bypass tube through one or more of the plurality of openings under peristalsis motion.

17. A gastrointestinal implant device configured for controlling absorption of nutrients, the device comprising:
(a) a float configured to be positioned within a stomach and selectively remain within the stomach by buoyant forces acting on the float, wherein the float comprises:
(i) two or more sections, wherein each of the sections comprise a first hollow interior configured to hold a fluid,
(ii) one or more connecting members, wherein each of the connecting members comprise a second hollow interior, wherein the one or more connecting members are configured to establish fluid communication between the two or more sections, such that the first hollow interior of each section in combination with the second hollow interior of each connecting member defines a continuous fluid channel,
(iii) a first opening at a distal end of the float, and
(iv) a second opening at a proximal end of the float, wherein the second opening is configured to receive consumed food from a lower esophagus sphincter; and
(b) a bypass tube configured to connect with the float at the distal end of the float, wherein the bypass tube comprises a plurality of openings and a plurality of curtains, wherein each of the curtains is operably configured to selectively cover one of the plurality of openings.

18. The device of claim 17, wherein the sections and connecting members are located adjacent one another in an alternating fashion thereby defining a continuous perimeter of the float.

19. The device of claim 17, wherein the continuous fluid channel is filled with air.

20. A method for assembling a device to control absorption of nutrients within a gastrointestinal tract using a transient implantable device, the method comprising:
(a) connecting a float of the device having a funnel shape with a bypass tube, wherein the float is configured to float upon gastric fluid within a stomach of the gastrointestinal tract;
(b) creating a plurality of openings within the bypass tube, such that the plurality of openings extend though an outer sidewall and an inner sidewall of the bypass tube to a hollow interior of the bypass tube, wherein the plurality of openings are spaced longitudinally along the bypass tube; and
(c) connecting a plurality of curtains to the bypass tube, wherein each of the plurality of curtains is configured to cover one of the plurality of openings, wherein the combination of the plurality of openings and the plurality of curtains together define a plurality of one-way openings along the bypass tube, wherein the plurality of one-way openings are configured to restrict food passing through the bypass tube from a proximal end to a distal end from exiting through any one of the plurality of one-way openings, wherein the plurality of one-way openings are further configured to permit digestive fluid within an intestine of the gastrointestinal tract to enter into the hollow interior of the bypass tube from outside of the bypass tube under peristalsis motion.
